(19) United States
(12) Patent Application Publication
(10) Pub. No.: US 2006/0167471 A1
(43) Pub. Date: Jul. 27, 2006

(54) Surgical marker

(75) Inventor: Michael Phillips, Oconomowoc, WI (US)

Correspondence Address: MICHAEL BEST & FRIEDRICH, LLP, 100 E WISCONSIN AVENUE, MILWAUKEE, WI, 53202, US

(73) Assignee: Vector Surgical, Madison, WI (US)

(21) Appl. No.: 11/341,288

(22) Filed: Jan. 27, 2006

Related U.S. Application Data
(60) Provisional application No. 60/647,652, filed on Jan. 27, 2005.

(51) Int. Cl. A61B 17/00

(52) U.S. Cl. 606/116

(57) ABSTRACT

A surgical instrument for use with a trocar that provides access to an interior portion of a patient. The instrument includes an elongated rod portion sized to fit within the trocar and a leg portion attached to the elongated rod portion and movable with respect to the leg portion to define an acute angle therebetween. A marker is attached to the leg portion and is operable to apply a marking substance to the interior of the patient.

### BACKGROUND

The present invention relates to a surgical marker. More particularly, the present invention relates to a surgical marker adapted to be inserted into a patient via a trocar to mark the interior of the patient.

During surgery, it is often necessary, or desirable, to mark the body of the patient. However, during laparoscopic procedures, this can be difficult. Thus, the surgeon relies on experience to determine size and position within the patient. For example, during a laparoscopic hernia repair a mesh is positioned to cover a defect. However, once the mesh is positioned, the mesh may obscure reference points typically used by the surgeon, thereby making it more difficult to properly position the mesh.

### SUMMARY

The present invention provides a marker for use inside the body of a patient. The marker is supported on the end of an elongated rod. A leg portion of the instrument is curved slightly to allow the proper positioning of the marker within the body. The marker and rod are sized to pass through a variety of trocars.

In one embodiment, the invention provides a surgical instrument for use with a trocar that provides access to an interior portion of a patient. The instrument includes an elongated rod portion sized to fit within the trocar and a leg portion attached to the elongated rod portion and movable with respect to the leg portion to define an acute angle therebetween. A marker is attached to the leg portion and is operable to apply a marking substance to the interior of the patient.

In another embodiment, the invention provides a laparoscopic surgical instrument usable during a surgical procedure in which at least one trocar provides access to an interior portion of a patient having a defect. The surgical instrument includes a marking device sized to pass at least partially through the trocar. The marking device includes a marker portion, a rod portion, and a leg portion. The leg portion supports the marker portion and is movable with respect to the rod portion to define a variable angle therebetween. A measuring device is sized to pass through the trocar such that the measuring device is positionable adjacent the defect and cooperates with the marking device to define a perimeter.

In still another embodiment, the invention provides a method of performing a laparoscopic procedure. The method includes positioning a trocar to provide access to an interior of a patient and passing a portion of a marking device through the trocar to position a leg portion within the interior. The leg portion defines an oblique angle with respect to a rod portion and supports a marker. In addition, the invention includes contacting the marker and the interior of the patient to make a visible mark, bending at least one of the leg portion and the rod portion to substantially align the leg portion and the rod portion along a common axis, and removing the marking device from the interior via the trocar.

### BRIEF DESCRIPTION OF THE DRAWINGS

FIG. 1 is a perspective view of an instrument suited for use in a laparoscopic procedure;

FIG. 2 is an enlarged perspective view of a portion of the instrument of FIG. 1, taken along line 2-2 of FIG. 1;

FIG. 3 is a schematic illustration of a patients torso during a laparoscopic procedure;

FIG. 4 is a perspective view of the instrument of FIG. 1 disposed within a trocar;

FIG. 5 is an interior view of a patient showing the instrument in use;

FIG. 6 is a measuring device for use with the instrument of FIG. 1; and

FIG. 7 is a side view of the instrument of FIG. 1 being removed or inserted through a trocar.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Laparoscopic surgery is becoming a more popular alternative to conventional open surgery. The minimally-invasive nature of laparoscopic surgery generally reduces the recovery time and the pain experienced by the patient. Laparoscopic techniques have been successfully applied to many procedures (e.g., hernia repair, gastric bypass, appendectomy, etc.). While laparoscopic surgery is applicable for many different procedures, the invention described herein will be discussed as being used in a hernia repair procedure. This should not be read as limiting the invention in anyway. Rather, the hernia repair is used for exemplary purposes.

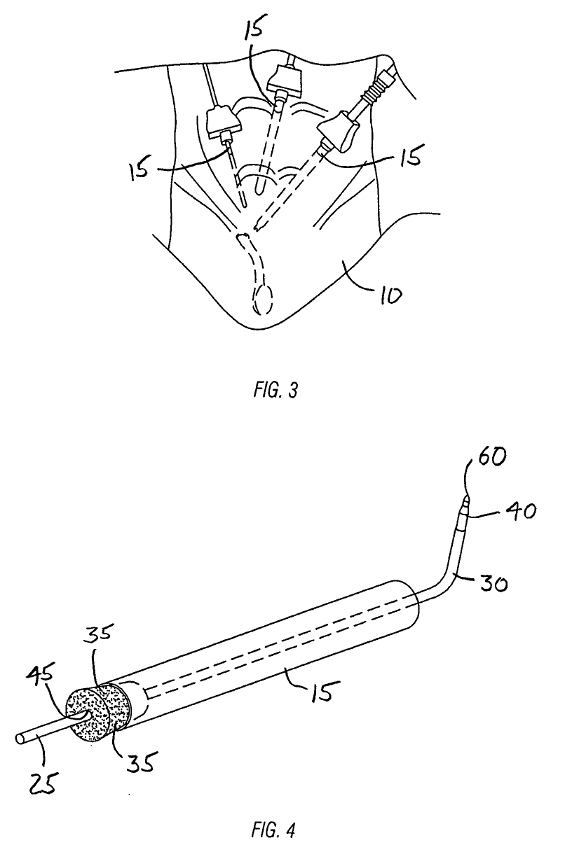

FIG. 3 illustrates the torso of a patient 10 undergoing a laparoscopic procedure. Typically, the surgeon places three small incisions in the patient to allow for the insertion of three trocars 15. Of course, other procedures may require fewer or more incisions and trocars 15. The trocars 15 are generally hollow tubes that allow for the insertion of various instruments. One instrument includes a miniature video camera that can be positioned by the surgeon to provide a view of the interior cavity of the patient 10. Generally, the video camera enlarges or magnifies the view to allow the surgeon to see all of the necessary details. The other two trocars 15 allow for the insertion and use of other instruments (e.g., scissors, forceps, graspers, and the like).

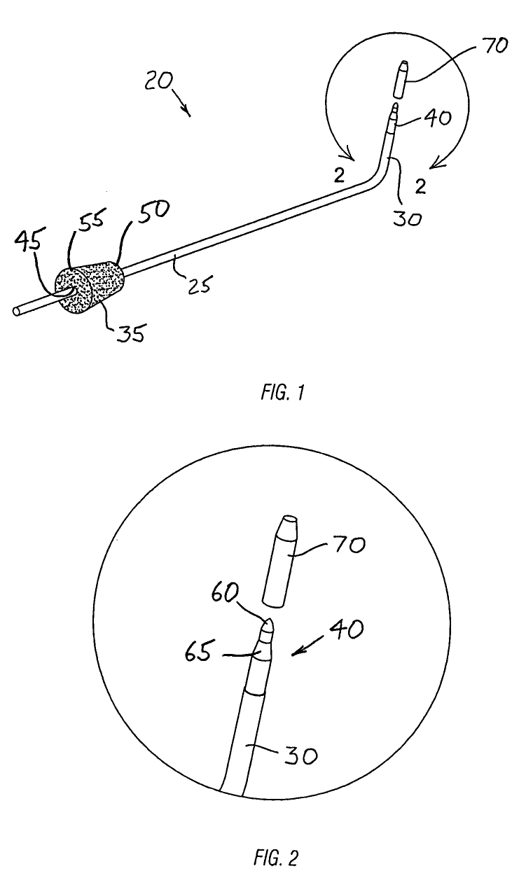

FIG. 1 illustrates one possible instrument 20 that can be inserted into one of the trocars 15. The instrument 20 includes an elongated rod portion 25, a leg portion 30, a stopper or adapter 35, and a marker 40. Generally, the elongated rod portion 25 and the leg portion 30 are integrally-formed as a single piece, preferably from a single material. However, other constructions may form the rod portion 25 and the leg portion 30 separately and then attach them to one another (e.g., adhesive, fasteners, welding, and the like).

The leg portion 30 is angled relative to the long axis of the elongated rod portion 25 to allow for the desired positioning of the marker 40. Specifically, the angle allows the surgeon to manipulate the marker 40 to mark interior surfaces of the patient 10. In preferred constructions, an acute or oblique angle between about zero and 90 degrees is defined. In more preferred constructions, the angle is between about 20 degrees and 70 degrees when in a non-deformed, or stress free condition. In some constructions, the leg portion 30 is articulated such that the surgeon can manipulate the angle between the leg portion 30 and the rod portion 25 during surgery. For example, one construction may include an inflatable bladder disposed within the leg portion 30 and/or the rod portion 25. When the bladder is inflated, the pressure forces the leg portion 30 to straighten relative to the rod portion 25. Careful control of the pressure within the bladder allows the surgeon to orient the leg portion 30 to almost any desired angle. In another construction, a cable, wire, shaft, or other linkage means engages the leg portion 30 and extends out of the rod portion 25 to provide access to the surgeon. In one example, the linkage means rotates a cam within or near the leg portion 30 that moves the leg portion 30 from an angled position to a position in which the leg portion 30 is substantially aligned with the rod portion 25. As one of ordinary skill will realize, there are many different ways to produce the desired articulated motion of the leg portion 30 relative to the rod portion 25. As such, the invention should not be limited to the few examples described herein.

In preferred constructions, a shape-memory material or superelastic material (e.g., titanium oxide, Nitinol, plastic, etc.) is employed to form the leg portion 30 or the leg portion 30 and the elongated rod portion 25 together. The superelasticity allows the angle of the leg portion 30 to change significantly relative to the rod 25 without permanently deforming the components. In still other constructions, a metal or plastic material is employed to form the rod 25 and/or the leg portion 30. Many different materials can be used to form the rod 25 and/or the leg portion 30 so long as the material is elastic enough to allow significant displacement of the leg portion 30 relative to the rod 25 without causing damage to the rod 25 or leg portion 30, and rigid enough to allow for manipulation and accurate positioning within the body of the patient 10. In one construction, plastic is used to achieve the desired results.

In most constructions, the rod 25 and leg portion 30 are smaller than the inside diameter of the smallest trocar 15 anticipated. For example, if a 5 mm trocar 15 is the smallest trocar 15 generally used, a rod 25 and leg portion 30 formed to be less than 5 mm in diameter will pass through the trocar 15. Of course, other constructions may employ a larger diameter rod 25 and leg portion 30, and be limited to use with larger trocars 15.

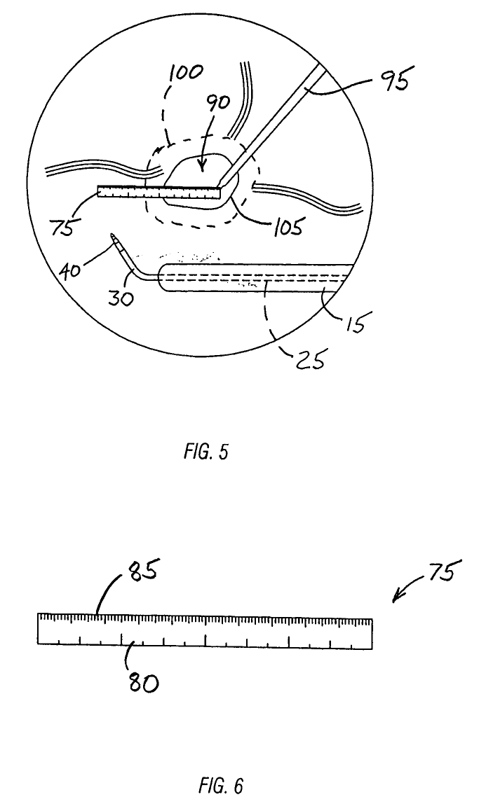

The stopper or adaptor 35 is generally frustoconical in shape and includes an opening, such as a central aperture 45. The central aperture 45 is sized to engage the elongated rod portion 25 and define a substantial airtight seal therebetween. The rod 25 is able to slide within the aperture 45 to move the marker 40 as necessary within the patient 10. It should be noted that while a centrally located aperture 45 is illustrated in FIGS. 1 and 4, there is no requirement that the aperture 45 be centrally located within the stopper 35.

The stopper or adaptor 35 includes a small diameter portion 50 that is smaller than the smallest trocar 15 that is anticipated, and a large diameter portion 55 that is larger than the largest diameter trocar 15 anticipated. For example, most trocars 15 are between 5 mm and 15 mm in diameter. Thus, one construction of a stopper 35 includes a small diameter portion 50 of about 3 mm and a large diameter portion 55 of about 17 mm. This stopper 35 will fit within any trocar 15 between 5 mm and 15 mm and will provide a seal between the outer surface of the stopper 35 and the inner surface of the trocar 15. Generally, a resilient material such as rubber (natural or synthetic), plastic, and the like, is employed to form the stopper 35. It should be noted that 5 mm trocars 15 are generally the smallest trocar 15 employed. Typically, 5 mm trocars 15 do not require a stopper or adaptor 35. As such, one preferred construction will employ a stopper having a small diameter portion 50 that is about 5 mm or greater. As one of ordinary skill in the art will realize, the small diameter portion 50 and the large diameter portion 55 are sized to engage and seal the trocar 15. As such, the sizes of the small diameter portion 50 and the large diameter portion 55 can vary greatly as can the size of the trocar 15 employed.

Turning to FIG. 2, the marker 40 and a portion of the leg portion 30 are shown enlarged. The marker 40 attaches to, or is formed as part of the leg portion 30 and extends along the long axis defined by the leg portion 30. The marker 40 includes a tip 60 and a reservoir 65 that holds a marking substance, such as ink. The ink flows from the reservoir 65 to the tip 60 and can be applied to the patient 10 as is well known in the art. Generally, the reservoir 65 is small, as the marker 40 is used once and discarded. However, some constructions may employ reusable markers 40 that require larger reservoirs 65. In most constructions, the reservoir 65 contains a surgical ink such as, but not limited to, gentian violet ink, or the like. The ink is preferably non-toxic, non-smearing, and non-irritating. In other constructions, other color inks, or other marking substances, may be employed as required for the particular procedure being performed. It should be noted that some constructions place all the needed ink in the tip 60 such that the tip 60 at least partially defines the reservoir 65.

In some constructions, a cap 70 is provided to cover the marker 40 before use of the instrument 20. The cap 70 inhibits contact between the marker tip 60 and other objects that may damage the tip 60 or deplete the ink supply. The cap 70 also inhibits drying of the tip 60, as often occurs when a marker tip 60 is left exposed to the air.

FIG. 4 shows the instrument 20 positioned within a trocar 15. As illustrated, the rod 25 is partially disposed within the trocar 15 such that the leg portion 30 extends beyond the end of the trocar 15 that is disposed within the patient 10 during the procedure. The rod 25 extends through the central aperture 45 of the stopper 35 such that the surgeon is able to manipulate the rod 25 (or the bladder, linkage means, etc.) to move the leg portion 30 and marker 40 from outside of the patient 10. The stopper 35 engages the end of the trocar 15 outside of the patient 10 and maintains a substantially air tight seal.

When the surgeon is using the instrument 20, it is sometimes necessary to make an accurate measurement within the patient. To that end, a measuring device 75, illustrated in FIG. 6, may be employed to allow for the accurate measurement of distance. The measuring device 75 is formed from a shape-memory material or superelastic material. Thus, the measuring device 75 can be rolled or deformed into a shape that will pass through one of the trocars 15, but that will not permanently deform the measuring device 75. The measuring device 75 can be unrolled within the patient and used to take measurements. In preferred constructions, the measuring device 75 is marked with both English units (inches) 80 and metric units (cm) 85. Of course, other constructions could use other measuring units as desired by the surgeon.

The use of the instrument 20 and the measuring device 75 will now be described as applied to a hernia repair procedure, and as illustrated in FIG. 5. FIG. 5 illustrates the view provided to the surgeon by the miniature camera positioned within the patient 10. As can be seen, the camera position is such that a defect 90 is centered in the surgeon's view. The surgeon inserts the measuring device 75 through one of the trocars 15 and holds it in the desired position using a grasper 95 that is inserted through the second trocar 15. The grasper 95 allows the surgeon to position the measuring device 75 as desired near the defect 90. The surgeon inserts the instrument 20 through the third trocar 15 and positions the stopper 35 in the trocar 15 to maintain a substantially air tight seal. The surgeon then manipulates the rod 25 (or the bladder, linkage means, etc.) to position the marker tip 60 as desired. The surgeon then uses the instrument 20 and the measuring device 75 in conjunction to place the desired markings on the patient 10 around the defect 90. For example, the surgeon may wish to mark a perimeter 100 around the defect 90 that is spaced a predefined distance (e.g., one inch, two inches, etc.) from an edge 105 of the defect 90.

Once the perimeter 100 is marked, the surgeon removes the measuring device 75 and the instrument 20 and inserts a mesh (not shown). The mesh is positioned over the defect 90 to repair the hernia. However, the mesh employed is generally opaque and as such blocks the surgeon's view of the defect 90. The surgeon is able to use the perimeter 100 marked using the instrument 20 to assure that the mesh is properly positioned over the defect 90.

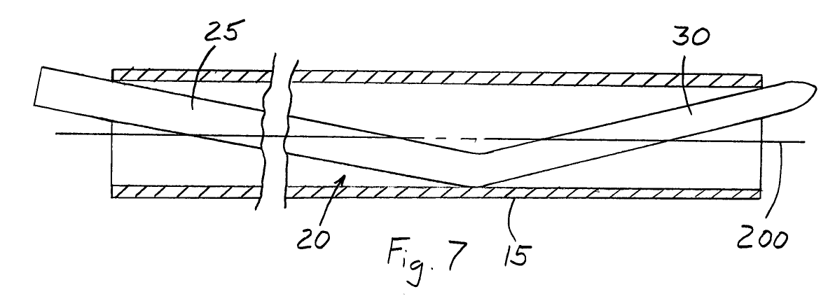

When inserting or removing the instrument 20, the leg portion 30 and/or the rod portion 25 are bent such that they are substantially aligned along a common axis 200. The amount of alignment required is only enough to allow the instrument to pass through the trocar 15, as illustrated in FIG. 7. Thus, it is not necessary that the leg portion 30 and rod portion 25 lie completely on the common axis 200, only that they sufficiently align with the common axis 200 to allow movement through the trocar 15.

As one of ordinary skill will realize, the instrument 20 described herein is suitable for use in virtually any laparoscopic procedure that could benefit from accurate markings placed within the body of the patient 10.

Generally, the instrument 20 (shown in FIGS. 1 and 2) is a single use item that is used during a laparoscopic surgical procedure. Thus, the instrument 20, including the ink within the marker 40, is sterilized before use. While many different sterilization procedures are possible, it is believed that gamma ray sterilization is best suited to the task of sterilizing the instrument 20, with other sterilization processes also being possible. In some constructions, the measuring device 75 is included with the instrument to define a kit. The kit is disposed within a container to provide storage of the instrument 20 and the measuring device 75 and to facilitate easy transfer of the kit from one location to another. For these constructions, the entire kit is sterilized before use and may be discarded after use, or reconditioned, resterilized, and reused.

Thus, the invention provides, among other things, an instrument 20 suited for use in marking the interior of a patient 10 during a laparoscopic procedure. More specifically, the invention provides a kit that allows for the accurate measurement and marking within a patient 10 during a laparoscopic procedure.

What is claimed is:

1. A surgical instrument for use with a trocar that provides access to an interior portion of a patient, the instrument comprising:
an elongated rod portion sized to fit within the trocar;
a leg portion attached to the elongated rod portion and movable with respect to the leg portion to define an acute angle therebetween; and
a marker attached to the leg portion and operable to apply a marking substance to the interior of the patient.

2. The instrument of claim 1, wherein the elongated rod portion and the leg portion are integrally-formed as a single piece using a shape-memory material.

3. The instrument of claim 1, wherein the acute angle is between about 20 degrees and 70 degrees.

4. The instrument of claim 1, wherein the leg portion is movable to vary the acute angle between zero degrees and 90 degrees without permanent deformation of the elongated rod portion and the leg portion.

5. The instrument of claim 1, further comprising an articulation member coupled the leg portion and adjustable to vary the acute angle.

6. The instrument of claim 5, wherein the articulation member includes an inflatable bladder.

7. The instrument of claim 1, wherein the marker includes a reservoir that contains a surgical ink.

8. The instrument of claim 7, wherein the marker further includes a tip that at least partially defines the reservoir.

9. A laparoscopic surgical instrument kit usable during a surgical procedure in which at least one trocar provides access to an interior portion of a patient, the surgical instrument comprising:
a marking device sized to pass at least partially through the trocar and including a marker portion, a rod portion, and a leg portion, the leg portion supporting the marker portion and movable with respect to the rod portion to define a variable angle therebetween; and
a measuring device sized to pass through the trocar such that the measuring device is positionable in the interior portion of the patient.

10. The laparoscopic surgical instrument kit of claim 9, wherein the rod portion and the leg portion are integrally-formed as a single piece.

11. The laparoscopic surgical instrument kit of claim 9, wherein the marker includes a reservoir that holds a marking substance and a tip that applies a portion of the marking substance to the interior of the patient.

12. The laparoscopic surgical instrument kit of claim 9, wherein the measuring device is elastically-deformable into a shape that allows for passage into the interior of the patient via the trocar.

13. The laparoscopic surgical instrument kit of claim 9, wherein the variable angle is variable between about zero degrees and ninety degrees.

14. The laparoscopic surgical instrument kit of claim 9, wherein the variable angle is between about 20 degrees and 70 degrees when the leg portion and the rod portion are in a stress free condition.

15. A method of performing a laparoscopic procedure, the method comprising:
positioning a trocar to provide access to an interior of a patient;
passing a portion of a marking device through the trocar to position a leg portion within the interior, the leg portion defining an oblique angle with respect to a rod portion and supporting a marker;
contacting the marker and the interior of the patient to make a visible mark;
bending at least one of the leg portion and the rod portion to substantially align the leg portion and the rod portion along a common axis; and
removing the marking device from the interior via the trocar.

16. The method of claim 15, further comprising bending at least one of the leg portion and the rod portion to substantially align the leg portion and the rod portion to facilitate insertion of the marking device into the trocar.

17. The method of claim 15, further comprising manipulating the rod portion from outside of the patient to move the marker.

18. The method of claim 15, further comprising additionally contacting the patient interior with the marker to mark a perimeter.

19. The method of claim 15, further comprising passing a measuring instrument through the trocar, positioning the measuring instrument adjacent a defect, and marking a perimeter a substantially fixed distance from the defect.

20. The method of claim 15, further comprising sterilizing the marking device.
